(19) United States
(12) Patent Application Publication
(10) Pub. No.: US 2022/0143239 A1
(43) Pub. Date: May 12, 2022

(54) FAR ULTRAVIOLET-C (UVC) 222 nm EXCIMER LAMP AND METHOD FOR ITS MANUFACTURE

(71) Applicant: PT. KENCANA INDAH PUTRA SAKTI, West Jakarta (ID)

(72) Inventors: Ferry Gunawan, West Jakarta (ID); Hendry Sunarjo, West Jakarta (ID); Yudhi Andrianto, West Jakarta (ID)

(73) Assignee: PT. KENCANA INDAH PUTRA SAKTI, West Jakarta (ID)

(21) Appl. No.: 17/522,945

(22) Filed: Nov. 10, 2021

(30) Foreign Application Priority Data
Nov. 11, 2020 (ID) P00202008447

(51) Int. Cl. A61L 2/10; A61L 2/00

(52) U.S. Cl. CPC A61L 2/10; A61L 2202/11; A61L 2/0047

(57) ABSTRACT

A cylindrical or flat far ultraviolet-C (UVC) 222 nm excimer lamp and a method for its manufacture are provided. The cylindrical or flat far UVC 222 nm excimer lamp can be used for safely sterilizing microorganisms (bacteria, fungi, or viruses) from the human body or otherwise, which can be used periodically or continuously. It comprises an anode inside of the lamp, a first insulator on the top of the anode, a second insulator above the first insulator with a height or distance, a connecting cover connecting the sides of the insulator the first with a second insulator for closing the gap or chamber, a valve on one side of the connecting cover for gas injection into the gap or chamber, and a cathode on the outside of the second insulator which is the same length and/or width as the anode.

### CROSS REFERENCE TO THE RELATED APPLICATIONS

This application is based upon and claims priority to Indonesian Patent Application No. P00202008447, filed on Nov. 11, 2020, the entire contents of which are incorporated herein by reference.

### TECHNICAL FIELD

The present invention relates to a far ultraviolet-C (UVC) 222 nm excimer lamp and a method of its manufacture. More particularly, the present invention discloses a far UVC 222 nm excimer lamp, which is safe for the human body and a method of its manufacture using electro-deposition process.

### BACKGROUND

Apparatus and methods for sterilizing microorganisms (bacteria, fungi, and viruses) periodically or continuously on products that are produced or contaminated have been widely disclosed in various articles or patent application documents and those granted a patent.

China's patent application No. CN 103 227 098 A, entitled “282 nm/222 nm Electrodeless Excimer Lamp”, discloses the field of optics in particular concerning the gas composition and gas pressure parameters of the electrodeless excimer lamp tube 282 nm waveband and 222 nm waveband capable of releasing electricity. And emit light through microwave excitation. The electrodeless light source has no pollution, has high light efficiency, has a long service life, and can expand the application range of exciter light sources in the printing field and photooxidation chloroethylene series hydrochloric ether saturation.

Korean Patent No. KR 102 092 339 B1, entitled “222 KrCl Method for Effective Microorganism Sterilization with The Intermittent Application of 222 KrCl Excimer Lamp Irradiation”, discloses a method for periodically sterilizing microorganisms using a 222 nm KrCl excimer lamp and, more specifically, an effective method of sterilizing microorganisms by overcoming the limitation of decreasing sterilization efficiency due to the filtering effect in high concentrations of contaminated water.

The present invention aims to provide a microorganism (bacteria, fungus, or virus) sterilization apparatus which is safe for humans and can be used periodically or continuously. In particular, the present invention provides a far UVC 222 nm excimer lamp, which is safe for the human body and a method for its manufacture by means of electro-deposition.

### SUMMARY

To overcome the problem of safe sterilization of objects and the human body, the present invention discloses an apparatus in the form of a far UVC 222 nm excimer lamp in various shapes (cylindrical or flat) and different sizes and in various UVC light emittance used for sterilizing microorganisms (bacteria, fungi, etc.) or viruses) from the human body safely or other objects, which can be used periodically or continuously.

The present invention also discloses a method for manufacturing a cylindrical or flat Far UVC 222 nm Used for sterilizing microorganisms (bacteria, fungi, or viruses) from the human body or other objects safely, which can be used periodically or continuously.

The far UVC 222 nm excimer lamp disclosed in the present invention also has several types with a said cylindrical or flat shape.

The type of far UVC 222 nm excimer lamp is distinguished by the number of insulators in the lamp, where the far UVC 222 nm excimer lamp with one insulator is called “Single Dielectric Barrier Discharge (SDBD)” and the far UVC 222 nm excimer lamp with two insulators is called “Double Dielectric Barrier Discharge (DDBD).”

The far UVC 222 nm excimer lamp, which can be cylindrical or flat, can be used used for sterilizing microorganisms (bacteria, fungi, or viruses) from the human body or other objects safely, which can be used periodically or continuously according to the present invention for the Single Dielectric Barrier Discharge type (SDBD), comprising:

an anode inside of the lamp with at least one connection point to a power supply;

a first insulator that is above the anode at a predetermined height or distance to form a gap or space;

a connecting cover to connect the first insulator side with the anode to cover the gap or chamber;

a valve located on one side of the connecting cover connecting the first insulator side to the anode for gas injection in the gap or chamber;

a cathode located on the outer surface of the first insulator with a length and/or width equal to the anode and having the pattern or shape of a wire mesh or other desired shape and having at least one point of connection to a negative power supply.

The cathode attached to the insulator of the far UVC 222 nm excimer lamp can be cylindrical or flat, which can have a pattern or shape of a wire mesh (square) or triangle, or a hexagon or other shape as desired.

The height or distance between the insulator and the anode to form a gap or space filled with gas in a cylindrical or flat far UVC 222 nm excimer lamp is 0.5 mm to 5 mm.

The insulator inside the far UVC 222 nm excimer lamp is cylindrical or flat (flat) is quartz or glass.

The cathode and anode of the far UVC 222 nm excimer lamp are cylindrical or flat with a thickness of 0.02 mm to 0.3 mm.

The cylindrical or flat far UVC 222 nm excimer lamp used for sterilizing microorganisms (bacteria, fungi, or viruses) from the human body or other objects safely, which can be used periodically or continuously according to the present invention for the Double Dielectric Barrier Discharge (DDBD) type, comprising:

an anode inside of the lamp with at least one point of connection to a power supply;

a first insulator at the top of the anode;

a second insulator which is above the first insulator by a predetermined height or distance to form a gap or space;

a connecting cover that connects the sides of the first insulator with the second insulator to cover the gap or space;

a valve located on one side of the connecting cover connecting the side of the first insulator to the second insulator (5) for gas injection in a gap or chamber;

a cathode on the outside of a second insulator which is the same length and/or width as the anode and has the pattern or shape of a wire mesh or other desired shape, and also has at least one point of connection to a negative power supply.

The cathode attached to the insulator bag of the far UVC 222 nm excimer lamp can be cylindrical or flat, which can have a pattern or shape of a wire mesh (square) or triangle, or a hexagon or other shape as desired.

The height or distance between the first insulator and the second insulator to form a gap or space filled with gas in a cylindrical or flat far UVC 222 nm excimer lamp is 0.5 mm to 5 mm.

Method for manufacturing a cylindrical or flat far UVC 222 nm excimer lamp for safely sterilizing microorganisms (bacteria, fungi, or viruses) from the human body or other objects, which can be used periodically or continuously according to the present invention for the Single Dielectric Barrier Discharge type. (SDBD), comprising:

forming an anode inside of the lamp with at least one point of connection to a positive output of power supply;

placing the first insulator above the anode with a predetermined height or distance to form a gap or space;

cover with a cover connecting the first insulator side with the anode side to cover the gap or space;

locating a valve on one side of the first insulator side connecting cover with the anode for gas injection in the gap or chamber;

forming a cathode on the outside of the first insulator, which is the same length and/or width as the anode and has a pattern or shape of wire mesh or other shapes as desired and also has at least one connection point to a negative electrical power supply;

where the cathode is formed through an electro-deposition process comprising:

melting the cathode base material to the outer surface of the first insulator (2) by connecting the positive output of power supply to the cathode base material and negative output of power supply to the outer surface of the first insulator through the electron-conducting medium to produce a cathode layer growing on the outer surface of the insulator,

covering with a mask the outer surface of the growth cathode layer to form the desired pattern or shape (such as wire mesh) on the growth cathode layer, which is above the outer surface of the first insulator,

dipping a layer of a growing electrode that has been coated with a mask into the etching solution with a predetermined time to remove the layer of a growing electrode that is not given a mask,

removing the mask from the growing electrode layer by dipping it into the etching solution so that the desired pattern or shape is formed on the cathode on the top surface of the first insulator.

Method for manufacturing a cylindrical or flat far UVC 222 nm excimer lamp used for sterilizing microorganisms (bacteria, fungi, or viruses) from the human body or other objects safely, which can be used periodically or continuously according to the present invention for the Double Dielectric Barrier Discharge type (DDBD), comprising:

forming an anode inside of the lamp with at least one point of connection to a positive output of power supply;

attaching the first insulator to the top of the anode;

placing the second insulator above the first insulator with a predetermined height or distance to form a gap or space;

covering the side of the first insulator and the second insulator with the connecting cover to cover the gap or space;

placing a valve on one side of the connecting cover connecting the first insulator to the second insulator for gas injection in a gap or chamber;

forming a cathode on the outside of the second insulator, which is the same length and/or width as the anode and has a pattern or shape of wire mesh or other shapes as desired and also has at least one connection point to a negative electrical power supply;

where the cathode is formed through an electro-deposition process comprising:

melting the cathode base material to the outer surface of the insulator by connecting the positive output of power supply to the cathode base material and negative output of power supply to the outer surface of the insulator through the electron-conducting medium to produce a cathode layer growing on the outer surface of the insulator,

covering with a mask the outer surface of the growth cathode layer to form the desired pattern or shape (such as wire mesh) on the growth cathode layer, which is above the outer surface of the insulator,

dipping a layer of a growing electrode that has been coated with a mask into the etching solution for a predetermined time to remove the layer of the growing electrode that is not given a mask,

removing the mask from the growing electrode layer by dipping it into the etching solution so that the desired pattern or shape is formed on the cathode above the insulator.

The anode and cathode of the Excimer Far UVC 222 nm cylindrical or flat lamp can be made of nickel, copper, silver, chromium, tungsten, or a combination thereof.

Masks are made of polymers soluble in polyurethane, cellulose, epoxy or can be melted by heat through the printing process.

The etching solution to remove the unmasked layer of the growing electrode can be FeCl₃, H2SO₄, HCl, HNO₃, or H₂O₂.

The etching solution to remove the mask from the growing electrode layer can be Na2SO4, NaNO3, NaCl, or NaOH.

### BRIEF DESCRIPTION OF THE DRAWINGS

The invention will be easy to understand by referring to the figures included in this description.

FIG. 1 shows the perspective of a cylindrical far UVC 222 nm excimer lamp, with 2 insulating tubes, according to an embodiment of the present invention.

FIG. 2 shows the perspective of a flat far UVC 222 nm excimer lamp with 2 insulating tubes, according to an embodiment of the present invention.

FIG. 3A and 3B shows a section of a cylindrical and flat far UVC 222 nm excimer lamp with 1 insulator, according to an embodiment of the present invention.

FIG. 4A and 4B show slices of a cylindrical or flat far UVC 222 nm excimer lamp with 2 insulators, according to an embodiment of the present invention.

FIG. 5 shows a method of manufacturing a far UVC 222 nm excimer lamp using an electro-deposition process to form a cathode adherent to a first insulator or a second insulator, according to an embodiment of the present invention.

### IN THE FIGURES

1 Cathode

2 First insulator

3 Anode

4 Valve

5 Second isolator

6 Height or distance between the anode and the first insulator

7 Height or distance between the second insulator and the first insulator

8 Connecting Cover

11 Power supply

12 Material of cathode

13 Medium for conducting atoms/electrons

14 The growing cathode layer

16 Masks

17 Media/mixture for the etcher

18 Media/mixture for etching

### DETAILED DESCRIPTION OF THE EMBODIMENTS

Various efforts have been made to overcome the spread of the virus that has become a pandemic using the 3M approach (wearing masks, maintaining distance, and washing hands) and spraying with disinfectant liquid on objects considered to be a place for the virus to survive.

The present invention discloses an apparatus in the form of a far UVC 222 nm excimer lamp with various shapes (cylindrical or flat) and different sizes and UVC light emittances. That excimer lamp is used for sterilizing microorganisms (bacteria, fungi, or viruses) from the human body or other objects safely, which can be used periodically or continuously.

The present invention also discloses a method for manufacturing cylindrical or flat far UVC 222 nm excimer lamp used for sterilizing microorganisms (bacteria, fungi, or viruses) from the human body or other objects safely, and which can be used periodically or continuously.

The far UVC 222 nm excimer lamp disclosed in the present invention also has cylindrical or flat shapes.

The type of far UVC 222 nm excimer lamp is distinguished by the number of insulators in the lamp. The far UVC 222 nm excimer lamp with one insulator is called “Single Dielectric Barrier Discharge (SDBD),” and the far UVC 222 nm excimer lamp with two insulators is called “Double Dielectric Barrier Discharge (DDBD).”

In order to make this invention easier to understand, the following disclosures or explanations disclosed concerning the figures included (attached).

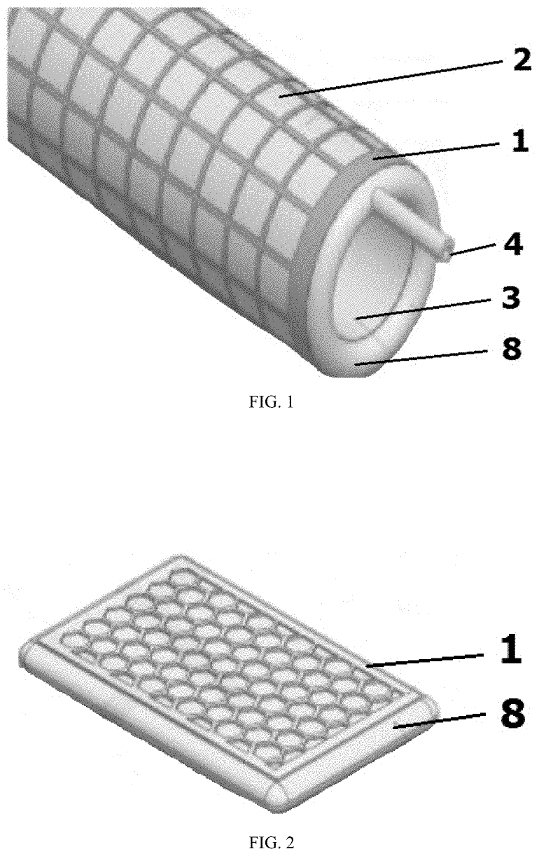

FIG. 1 shows a perspective view of a cylindrical far UVC 222 nm excimer lamp, with 2 insulating tubes, according to an embodiment of the present invention.

FIG. 1 shows an anode (3) in the form of an elongated tube or a cylindrical shape located in the innermost part of a far UVC 222 nm excimer lamp. On top of the anode is placed a first insulator (5) (not shown) then a second insulator (2). The first insulator (5) and the second insulator has a predetermined height or distance to form a gas chamber or gap. Above the second insulator is placed a cathode (2) which has the shape of a wire mesh.

FIG. 2 showing the perspective of a flat far UVC 222 nm excimer lamp with 2 insulating tubes, according to an embodiment of the present invention.

In FIG. 2, the cathode (1), which is tightly attached to the outer surface of the first insulator (2), and the connecting cover (8), which covers the gap or space between the first insulator (2) and the second insulator (5) (not shown).

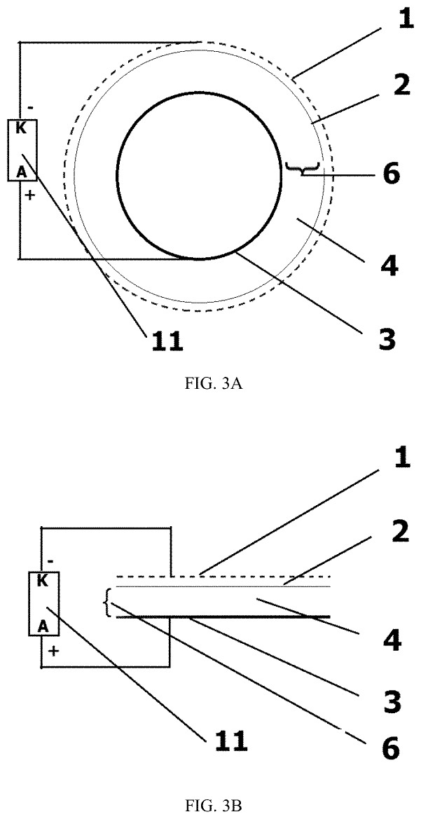

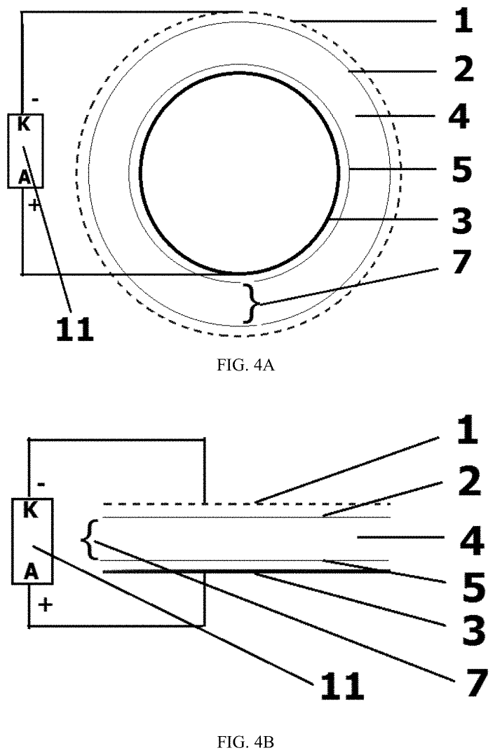

Referring to FIG. 3A and 3B shows a section of a cylindrical and flat far UVC 222 nm excimer lamp with 1 insulator or Single Dielectric Barrier Discharge (SDBD) type, according to an embodiment of the present invention.

A cylindrical or flat far UVC 222 nm excimer lamp used for sterilizing microorganisms (bacteria, fungi, or viruses) from the human body or other objects safely, which can be used periodically or continuously according to the present invention for the Single Dielectric Barrier Discharge type (SDBD), comprising:

an anode (3) inside of the lamp with at least one connection point to a positive output of power supply (11);

a first insulator (2) located above the anode (1) at a predetermined height or distance (6) to form a gap or space;

a connecting cover (8) for connecting the first insulator side (2) with the anode (3) for closing the gap or chamber;

a valve (4) located on one side of the connecting cover (8) connecting the first insulator side (2) with the anode (1) for gas injection into the gap or chamber;

a cathode (1) located on the outer surface of the first insulator (2) with a length and/or width equal to the anode (3) having the pattern or shape of a wire mesh or other desired shape and also having at least one connection point to a negative output of power supply (11).

The cathode (1) attached to the first insulator (2) of the cylindrical or flat cylindrical far UVC 222 nm excimer lamp may have a wire mesh pattern or shape (rectangular or triangular, or hexagonal) or other shapes.

The height or distance (6) between the first insulator (2) and the anode (3) to form a gap or space filled with gas in a cylindrical or flat far UVC 222 nm excimer lamp is 0.5 mm to 5 mm.

The first insulator (2) in the cylindrical or flat far UVC 222 nm excimer lamp is quartz or glass.

The cathode and anode of the far UVC 222 nm excimer lamp are cylindrical or flat with a thickness of 0.02 mm to 0.3 mm.

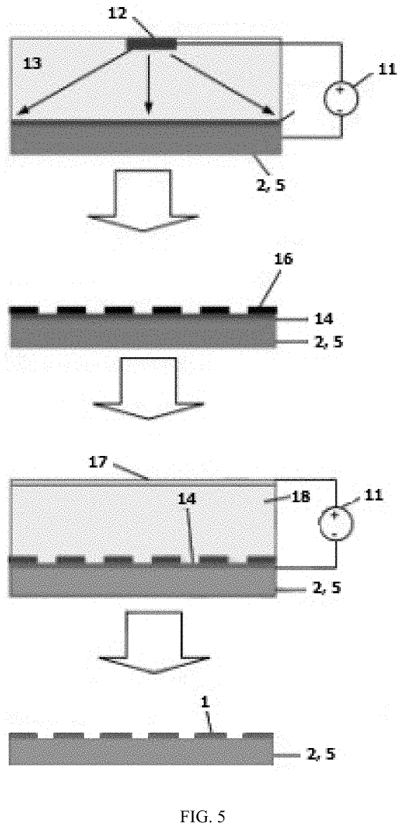

FIGS. 4A and 4B show a section of a cylindrical or flat far UVC 222 nm excimer lamp with 2 insulators or Double Dielectric Barrier Discharge (DDBD) type, according to an embodiment of the present invention.

A cylindrical or flat far UVC 222 nm excimer lamp, used for sterilizing microorganisms (bacteria, fungi, or viruses) from the human body or other objects safely, which can be used periodically or continuously according to the present invention for the Double Dielectric Barrier Discharge type (DDBD), comprising:

an anode (3) inside of the lamp with at least one connection point to a positive output of power supply (11);

a first insulator (2) at the top of the anode (3);

a second insulator (5) which is above the first insulator (2) by a predetermined height or distance (7) to form a gap or space;

a connecting cover (8) connecting the sides of the first insulator (2) with the second insulator (5) to cover the gap or space;

a valve (4) located on one side of the connecting cover (8) of the first insulator side (2) with the second insulator (5) for gas injection in a gap or chamber;

a cathode (1) located on the outer surface of the first insulator (2) with a length and/or width equal to the anode (3) having the shape of a wire mesh or other desired shape and also having at least one point of connection to a negative output of power supply (11).

The cathode (1) attached to the first insulator (2) of the cylindrical or flat cylindrical far UVC 222 nm excimer lamp (flat) may have a wire mesh pattern or shape (rectangular or triangular, or hexagonal) or other shapes.

The height or distance (7) between the first insulator (2) and the second insulator (5) to form a gap or space filled with gas in a cylindrical or flat 222 nm Excimer Far UVC Lamp is 0.5 mm to 5 mm.

The first insulator (2) and the second insulator (5) located inside the cylindrical or flat far UVC 222 nm excimer lamp are made from quartz or glass.

The cathode and anode of the far UVC 222 nm excimer lamp are cylindrical or flat with a thickness of 0.02 mm to 0.3 mm.

Referring to FIG. 5, which shows a method of manufacturing a far UVC 222 nm excimer lamp using an electro-deposition process to form a cathode adherent to a first insulator or a second insulator, according to an embodiment of the present invention.

A method for manufacturing a cylindrical or flat far UVC 222 nm excimer lamp, used for sterilizing microorganisms (bacteria, fungi, or viruses) from the human body or other objects safely, which also can be used periodically or continuously to the present invention for the Single Dielectric Barrier Discharge(SDBD) type, comprising:

forming an anode (3) inside of the lamp with at least one connection point to a positive output of power supply (11);

placing the first insulator (2) above the anode (3) at a predetermined height or distance (6) to form a gap or space;

covering the first insulator side (2) with the anode side (3) with a connecting cover (8) to cover the gap or chamber;

locating a valve (4) on one side of the connecting cover (8) the first insulator side (2) with the anode (3) for gas injection in the gap or chamber;

forming a cathode (1) on the outside of the first insulator (2) which is the same length and/or width as the anode (3) and has the pattern or shape of a wire mesh or other shape as desired and also has at least one connection point to a negative output of power supply (11);

wherein the cathode (1) is formed by an electro-deposition process comprising:

melting the cathode base material (12) onto the outer surface of the first insulator (2) by connecting the positive output of power supplies (11) to the cathode base material (12) and the negative output of power supplies (11) to the outer surface of the first insulator (2) through an electron-conducting medium (13) resulting in a growing cathode layer (14) on the outer surface of the first insulator (2),

coating with a mask (16) the outer surface of the grow cathode layer (14) to form a desired pattern or shape (such as wire mesh) on the grow cathode layer (14), which is above the outer surface of the first insulator (2),

dipping the growing electrode layer (14), which has been coated with a mask (16) into the etching solution for a predetermined time to remove the unmasked growing electrode layer (14),

removing the mask (16) from the growing electrode layer (14) by dipping it into the etching solution so that the desired pattern or shape is formed on the cathode (1) above the first insulator (2).

Method for manufacturing a cylindrical or flat far UVC 222 nm excimer lamp used for sterilizing microorganisms (bacteria, fungi, or viruses) from the human body or other objects safely, which can be used periodically or continuously according to the present invention for the Double Dielectric Barrier Discharge (DDBD) type, comprising:

forming an anode (3) inside of the lamp with at least one connection point to a positive output of power supply (11);

attaching the first insulator (2) to the top of the anode (3);

placing the second insulator (5) above the first insulator (2) with a predetermined height or distance (7) to form a gap or space;

covering with a connecting cover (8) the side of the first insulator (2) and the second insulator (5) to cover the gap or space;

placing a valve (4) on one side of the connecting cover (8) connecting the first insulator (2) with the second insulator (5) for gas injection in a gap or chamber;

forming a cathode (1) on the outside of the second insulator (5) which is the same length and/or width as the anode (3) and has the pattern or shape of a wire mesh or other shape as desired and also has at least one connection point to a negative output of power supply (11);

wherein the cathode (1) is formed by an electro-deposition process comprising:

melting the cathode base material (12) onto the outer surface of the first insulator (2) by connecting the positive output of power supplies (11) to the cathode base material (12) and the negative output of power supplies (11) to the outer surface of the first insulator (2) through an electron-conducting medium (13) resulting in a growing cathode layer (14) on the outer surface of the first insulator (2),

coating with a mask (16) the outer surface of the grow cathode layer (14) to form a desired pattern or shape (such as wire mesh) on the grow cathode layer (14), which is above the outer surface of the first insulator (2),

dipping the growing electrode layer (14), which has been coated with a mask (16) into the etching solution for a predetermined time to remove the unmasked growing electrode layer (14)

remove the mask (16) from the growing electrode layer (14) by dipping it into the etching solution so that the desired pattern or shape is formed on the cathode (1) above the first insulator (2).

The anode and cathode of the cylindrical or flat excimer far UVC 222 nm lamp may be made from nickel, copper, silver, chromium, tungsten, or a combination thereof.

Masks are made of polymers soluble in polyurethane, cellulose, epoxy, or melt by heat through the printing process.

The etching solution to remove the unmasked layer of the growing electrode can be FeCl₃, H₂SO₄, HCl, HNO₃, or H₂O₂.

The etching solution to remove the mask from the growing electrode layer can be Na₂SO₄, NaNO₃, NaCl, or NaOH.

What is claimed is:

1. A cylindrical or flat far ultraviolet-C (UVC) 222 nm excimer lamp periodically or continuously used for sterilizing microorganisms (bacteria, fungi, or viruses) from a human body or other objects safely, comprising:
an anode inside of the cylindrical or flat far UVC 222 nm excimer lamp with at least one connection point to a positive output of a power supply;
a first insulator located above the anode at a predetermined height or distance to form a gap or space;
a connecting cover for connecting a first insulator side with the anode to cover the gap or chamber;
a valve located on one side of the connecting cover connecting the first insulator side with the anode for gas injection into the gap or chamber; and
a cathode located on an outer surface of the first insulator with a length and/or width equal to the anode having a shape of a wire mesh or other desired shape and also having at least one connection point to a negative output of the power supply.

2. The cylindrical or flat far UVC 222 nm excimer lamp according to claim 1, wherein the cathode is in a form of a wire mesh (square, triangle, hexagon) or any other desired shape.

3. The cylindrical or flat far UVC 222 nm excimer lamp according to claim 1, wherein the predetermined height or distance between the first insulator and the anode to form the gap or space is 0.5 mm to 5 mm.

4. The cylindrical or flat far UVC 222 nm excimer lamp according to claim 1, wherein the first insulator is quartz or glass.

5. The cylindrical or flat far UVC 222 nm excimer lamp according to claim 1, wherein the cathode and the anode have a thickness of 0.02 mm to 0.3 mm.

6. A cylindrical or flat far ultraviolet-C (UVC) 222 nm excimer lamp periodically or continuously used for sterilizing microorganisms (bacteria, fungi, or viruses) from a human body or other objects safely, comprising:
an anode inside of the cylindrical or flat far UVC 222 nm excimer lamp with at least one connection to a positive output of a power supply;
a first insulator at a top of the anode;
a second insulator located above the first insulator with a predetermined height or distance to form a gap or space;
a connecting cover for connecting sides of the first insulator with the second insulator to cover the gap or space;
a valve located on one side of the connecting cover of a first insulator side with the second insulator for gas injection in a gap or chamber; and
a cathode located on an outer surface of the first insulator with a length and/or width equal to the anode having a shape of a wire mesh or other desired shape and also having at least one point of connection to a negative output of the power supply.

7. The cylindrical or flat far UVC 222 nm excimer lamp according to claim 6, wherein the cathode is in a form of a wire mesh (triangle, rectangular, hexagon) or any other desired shape.

8. The cylindrical or flat far UVC 222 nm excimer lamp according to claim 6, wherein the predetermined height or distance between the second insulator and the first insulator to form the gap or space is 0.5 mm to 5 mm.

9. The cylindrical or flat far UVC 222 nm excimer lamp according to claim 6, wherein the first insulator and the second insulator are quartz or glass.

10. The cylindrical or flat far UVC 222 nm excimer lamp, according to claim 6, wherein the cathode and the anode have a thickness of 0.02 mm to 0.3 mm.

11. A method for manufacturing a cylindrical or flat far ultraviolet-C (UVC) 222 nm excimer lamp periodically or continuously used for sterilizing microorganisms (bacteria, fungi, or viruses) from a human body or other objects safely, comprising:
forming an anode inside of the cylindrical or flat far UVC 222 nm excimer lamp with at least one connection point to a positive output of a power supply;
placing the first insulator above the anode at a predetermined height or distance to form a gap or space;
covering a first insulator side with an anode side with a connecting cover to cover the gap or chamber;
locating a valve on one side of the connecting cover of the first insulator side that connects with the anode for gas injection in the gap or chamber;
forming a cathode on an outside of the first insulator of a same length and/or width as the anode having a shape of a wire mesh or other desired shape and also having at least one point of connection to a negative output of the power supply;
wherein the cathode is formed by an electro-deposition process, comprising:
melting a cathode base material onto an outer surface of the first insulator by connecting the positive output of the power supply to the cathode base material and the negative output of the power supply to the outer surface of the first insulator through an electron-conducting medium resulting in a growing cathode layer on the outer surface of the first insulator,
coating with a mask an outer surface of the growing cathode layer to form a desired pattern or shape (such as wire mesh) on the growing cathode layer, wherein the growing cathode layer is above the outer surface of the first insulator,
dipping a growing electrode layer, wherein the growing electrode layer has been coated with the mask into an etching solution for a predetermined time to remove an unmasked growing electrode layer, and
removing the mask from the growing electrode layer by dipping the growing electrode layer into the etching solution, wherein the desired pattern or shape is formed on the cathode above the first insulator.

12. The method for manufacturing the cylindrical or flat far UVC 222 nm excimer lamp according to claim 11, wherein the cathode and the anode are nickel, copper, silver, chromium, tungsten, or a combination thereof.

13. The method for manufacturing the cylindrical or flat far UVC 222 nm excimer lamp according to claim 11, wherein the mask is made of a soluble polymer in polyurethane, cellulose, epoxy, or a polymer that is heat-meltable by a printing process.

14. The method for manufacturing the cylindrical or flat far UVC 222 nm excimer lamp according to claim 11, wherein the etching solution is FeCl₃, H₂SO₄, HCl, HNO₃, or H₂O₂.

15. The method for manufacturing the cylindrical or flat far UVC 222 nm excimer lamp according to claim 11, wherein the etching solution is NaSO₄, NaNO₃, NaCl, or NaOH.

16. A method for manufacturing a cylindrical or flat far ultraviolet-C (UVC) 222 nm excimer lamp periodically or continuously used for sterilizing microorganisms (bacteria, fungi, or viruses) from a human body or other objects safely, comprising:
forming an anode inside of the cylindrical or flat far UVC 222 nm excimer lamp with at least one connection point to a positive output of a power supply;
attaching a second insulator to an upper surface of the anode;
placing a first insulator above the second insulator with a predetermined height or distance to form a gap or space;
covering a side of the second insulator with the first insulator with a connecting cover to cover the gap or space;
placing a valve on one side of the connecting cover connecting the second insulator with the first insulator for gas injection into the gap or chamber;
forming a cathode on an outside of the first insulator with a length and/or width equal to the anode having a shape of a wire mesh or other desired shape and also having at least one point of connection to a negative output of the power supply;
wherein the cathode is formed by an electro-deposition process comprising:
melting a cathode base material onto an outer surface of the first insulator by connecting a positive output of power supplies to the cathode base material and a negative output of power supplies to the outer surface of the first insulator through an electron-conducting medium resulting in a growing cathode layer on the outer surface of the first insulator,
coating with a mask an outer surface of the growing cathode layer to form a desired pattern or shape (such as wire mesh) on the growing cathode layer, wherein the growing cathode layer is above the outer surface of the first insulator,
dipping a growing electrode layer, wherein the growing electrode layer has been coated with the mask into an etching solution for a predetermined time to remove an unmasked growing electrode layer, and
removing the mask from the growing electrode layer by dipping the growing electrode layer into the etching solution, wherein the desired pattern or shape is formed on the cathode above the first insulator.

17. The method for manufacturing a cylindrical or flat far UVC 222 nm excimer lamp according to claim 16, wherein the cathode and the anode are nickel, copper, silver, chromium, tungsten, or a combination thereof.

18. The method for manufacturing a cylindrical or flat far UVC 222 nm excimer lamp according to claim 16, wherein the mask is made of a soluble polymer in polyurethane, cellulose, epoxy, or a polymer that is heat-meltable by a printing process.

19. The method for manufacturing a cylindrical or flat far UVC 222 nm excimer lamp according to claim 16, wherein the etching solution is FeCl₃, H₂SO₄, HCl, HNO₃, or H₂O₂.

20. The method for manufacturing a cylindrical or flat far UVC 222 nm excimer lamp according to claim 16, wherein the etching solution is NaSO₄, NaNO₃, NaCl, or NaOH.
